(19) United States
(12) Patent Application Publication
(10) Pub. No.: US 2023/0240826 A1
(43) Pub. Date: Aug. 3, 2023

(54) PROCEDURE FOR REPAIRING AND TREATING PELVIC PROLAPSE IN A FEMALE PATIENT, A MEDICAL INSTRUMENT FOR PERFORATION OF THE CERVIX, A PROCEDURE FOR REMOVING TENDON TISSUE OF A TENDON FROM A HUMAN OR ANIMAL BODY, AND A PROCEDURE FOR THE PREPARATION OF TENDON TISSUE

(71) Applicant: Amadeus Hornemann, Frankfurt (DE)

(72) Inventor: Amadeus Hornemann, Frankfurt (DE)

(21) Appl. No.: 17/590,312

(22) Filed: Feb. 1, 2022

(51) Int. Cl. A61F 2/00; A61B 17/42; A61B 17/06; A61B 17/04

(52) U.S. Cl. CPC A61F 2/0045; A61B 17/4241; A61B 17/06; A61B 17/0485; A61F 2220/0075

(57) ABSTRACT

A procedure for repairing and treating pelvic prolapses in a female patient, whereas the pelvic organs include vagina, cervix, uterus, bladder, urethra and rectum, comprises the following steps: providing an elongated tendon-type implant having a first end and a second end, attaching the first end of the implant to the longitudinal ligament of the patient, attaching the second end of the implant to the pelvic organ of the patient, wherein the attachment of the implant is to be made under tension so that the pelvic organ is raised from the prolapsed position to a normal position.

### BACKGROUND OF THE INVENTION

Pelvic organ prolapse is a very common diagnosis for women, not only for elder women. The pelvic organ prolapse condition is a descending of pelvic organs, like vagina and/or bladder and/or uterus and/or rectum, from their normal position. The prolapse can reach severe stages when the organs descend so far as they tend to protrude through the vulvo-vaginal opening. This phenomenon results from weakening of the supportive, cohesive and organ-suspending tissue. Risk factors are for example vaginal deliveries, weak connective tissue, age and other medical conditions such as obesity.

The symptoms associated with prolapse are variable and may in particular include feelings of pelvic heaviness, pelvic lumbar pain, foreign body sensation, obstructive urinary symptoms with or without urinary or fecal incontinence. The seriousness of pelvic organ prolapse is especially evaluated in relation to its impact on patients' quality of life in terms of functional discomfort.

Pelvic organ prolapse is a frequent pathology whose prevalence is estimated at between 30 and 50% of women irrespective of the type of prolapse and/or the stage thereof.

Treatment include conservative and interventional options. Conservative treatment is physiotherapy with pelvic training like kegel exercises and a therapy with pessaries (e.g. plastic cube, ring and other foreign bodies being placed in the vagina to prevent the structures from descending).

If a conservative therapy is insufficient, numerous surgical techniques for treating prolapse are available consisting of inserting implants or prostheses to give support to the prolapsed organ. These options often include multiple ways of hysterectomy often combined with anterior and posterior colporrhaphy, fixation of the cervix or vagina to the spine or to ligaments etc. Access routes are through the vagina, a laparotomy or minimally invasive procedures, for instance laparoscopy and robotic surgery.

In the past most of the operative procedures with native tissue did not show good long-term results. Therefore, bio compatible plastic meshes were introduced for implantation.

U.S. Pat. No. 6,592,515B2 discloses a procedure for surgical treatment of pelvic floor disorders with a preassembled implantable article. The implants are described as preferably constructed of mesh material.

WO2010087923 discloses a medical article comprising multiple mesh flaps used to repair prolapses of various pelvic organs including vaginal vault prolapse.

US 2008/0021265 A1 discloses a prosthesis to be implanted in a Human body for repairing pelvic organ prolapses in a female patient and a surgical procedure using such a prosthesis. Wherein this prosthesis is comprised of a plastic mesh material.

### DISCLOSURE

### Problem to be Solved

Short- and long-term results of meshes were convincing at first but after a while, severe side effects were seen. In addition, a complete removal of the inserted material is usually almost impossible. In addition, many health insurances will no more cover costs for the treatment of side effects of implanted meshes. At least some of the manufacturers of plastic meshes have now withdrawn their products from the market; also because of a class action lawsuit by thousands of affected women.

Alternative procedures and materials for surgical of treatment pelvic organ prolapse are urgently needed.

### Solution of the Problem

### First Aspect

According to a first aspect, the invention relates to a procedure for repairing and treating pelvic prolapses in a female patient, whereas the pelvic organs include vagina, cervix, uterus, bladder, urethra and rectum, comprises the following steps:

providing an elongated tendon-type implant having a first end and a second end,

attaching the first end of the implant to the longitudinal ligament of the patient,

attaching the second end of the implant to the pelvic organ of the patient,

wherein the attachment of the implant is to be made under tension so that the pelvic organ is raised from the prolapsed position to a normal position.

The elongated tendon-like shaped implant, preferably consisting of endogenous or exogenous tendon tissue or bio compatible material, provide benefits compared to mesh or mesh-like implants. Such implants enable a more targeted use on the prolapsed organs in the abdomen for the repair of pelvic organ prolapses. In addition, they reduce the side effects and problems after the surgery, if side effects occur the removal would still be possible, because the ingrowth is reduced through the composition of the implant. If even an endogenous tendon tissue implant is used, then the side effects will be reduced even further as the acceptance of this material inside the body will be maximized.

The attaching of the first end and/or the second end of the implant according to step b) and c) may be performed by fastening elements such as, but not limiting to, sutures, screws or staples.

An advantageous embodiment provides a tunnel with a tunnel entrance and a tunnel exit is formed in or around the longitudinal ligament in the region of the promontory ossis sacri, wherein the attachment according to step b) is performed by passing one end of the implant through the tunnel. By forming and using a tunnel, fastening elements can be avoided and the amount of exogenous materials reduced.

The tunnel may inserted in such a way, that the tunnel entrance and the tunnel exit are provided on the side of the longitudinal ligament facing away from the promontory ossis sacri. To provide an optimal path of force for the implant during the raising of the prolapsed organs and in the later fixed position, and to avoid stress and injury to surrounding tissue.

Furthermore, it is advantageous if at least one perforation is made in the cervix, wherein the attaching to the cervix according to step c) is performed by passing one end of the implant through the perforation. Preferably this perforation is performed in the direction caudal from ventral to dorsocranial. This direction, with the described entry point and exit point, is preferred, as it minimizes the risk of injury to other pelvic organs such as the bladder while performing the perforation.

It has been shown that it is particularly advantageous when two parallel or almost parallel perforations are performed, preferably with an angular deflection of 0° to 30° to each other, wherein each perforation have a cranial and a caudal opening. The parallel or near parallel perforations provide preferable force induction into the cervix from the implant and the tensile force applied.

It is also advantageous if the attaching to the cervix according to step c) is performed as follows: inserting one end of the implant through the cranial opening of the first perforation in direction to the caudal opening and passing in caudal direction through the caudal opening, after exiting the caudal opening of the first perforation inserting the end of the implant into the caudal opening of the second perforation and passing in cranial direction through the cranial opening. In this case the shape, direction and routing of the perforations used is especially beneficial to provide preferable force induction from the implant to the cervix to avoid kinking of the cervix while being raised out of the prolapsed position and remaining fixed in the normal position.

It is further advantageous if the ends of the implant are pulled together after attaching the tendon tissue in step b) and c) to provide the tension according to step d), to provide the tensile force to raise the attached prolapsed organ into the normal position.

It is also advantageous if the ends of the implant are sutured together under tension applied in step d), so that the raised organ will stay fixed in the normal position.

The implant may be made of biocompatible material and/or of biological material. The use of biocompatible or endogenous materials, in particular tendon tissue material, for implantation articles in prolapse repair surgeries would overcome the disadvantages of meshes in use and provide a much needed new solution.

It has turned out to be particularly advantageous when the provided implant is composed from prepared tendon tissue from the semitendinosus tendon or the gracilis tendon of the patient, as this tendon is harvested for some other medical procedures and thus a wealth of experience in the medical field has been accumulated. Furthermore, this tendon can be harvested, whereby the functionality is restored after some time due to fusion of the attached muscle with surrounding muscles.

The procedure for repairing and treating pelvic prolapses is preferably performed laparoscopically, to reduce the visible surgery scars to a minimum.

The first aspect has alternative procedures depending on the condition or the preferences of the patient.

The procedure can be performed with or without a supracervical hysterectomy. Thus it is also suitable for patients planning a pregnancy in the future or with the desire to keep the uterus.

There are also cases where hysterectomy with the removal of the uterus and cervix has been already performed in a different surgery or the cervix needed to be removed in this procedure. This procedure can still be performed with variations. As the uterus or cervix are no longer available, and thus no perforations can be made, the implant will be attached to the vaginal wall and tautly dawn to repair the prolapse.

### Second Aspect

According to a second aspect, the invention relates to a medical instrument, which is preferably used for perforation of the cervix as described in the first aspect of the invention. Said instrument being realized as a monopolar spatula and having a kink at a distance kink from the cutting end, in particular for use in the above described procedure for realizing the perforation of the cervix.

The kink may preferably enclose an angle α in the range of 90° to 175°, and preferably in the range of 120° to 150°. This kink may preferably be located in a distance of 2 to 5 cm from the cutting end of the spatula, and preferably in the range of 3 to 4 cm. The width of the cutting end is preferably 3 to 5 mm, preferably 4 mm. The thickness of the cutting end is preferably 2 mm. The instrument preferably exhibits this kink only in the introduced state and not during introduction into the body of the patient. The cutting end of the Instrument is preferably of a round shape when viewed from above, view direction orthogonal to the longitudinal axis of the instrument. This instrument enables the perforations in described shape and routing, as the entry point of the perforation in the cervix is not reachable with a straight spatula.

### Third Aspect

As already mentioned when explaining the first embodiment, it is advantageous if tendon material is used as the implant when repairing and treating pelvic prolapses according to the first aspect of the invention.

In the medical field of orthopedic surgeries, the semitendinosus tendon is the standard material for anterior cruciate ligament replacement. It has been in use for decades for this indication. The morbidity is very low and usually people can compensate the loss of this tendon without or minimal a problem to the leg.

However, persons skilled in the art have not yet recognized the usage of tendon material for the purpose of repairing and treating pelvic prolapses could be advantageous.

According to the third aspect of the invention, a procedure for removing tendon tissue of a tendon from a human or animal body was found comprising the following steps: a) exposure of a tendon present in the human or animal body,

dividing the tendon after exposing it in the direction of its longitudinal fibers into two elongated portion of tendon tissue, separation and removal of one of the two portions of tendon tissue; and remaining the other portion of the tendon tissue in the body so that it continues to provide its function and the function of the muscle associated with it.

Furthermore it was found, that the removed tendon tissue portion comprises only ¼ to ¾, preferably ¼ to ½, of the cross-sectional area of the exposed tendon. It is advantageous that at least ¼ of the tendon cross-sectional area remains in the leg for the patient's mobility and fast recovery.

It is advantageous when dividing the exposed tendon according to step b) and c) is performed by first slitting the tendon and then dividing the slitted tendon with a tendon stripper, preferably an open tendon stripper. The use of an open tendon stripper enables inserting the stripper into the first made short division of the tendon.

It has turned out to be particularly advantageous when the exposed tendon is the semitendinosus tendon and the removed tendon tissue portion is tissue of the semitendinosus tendon. The semitendinosus tendon is good accessible and therefore well suited for a harvest. Material for a later implantation in the patient's body for best results with regard to later problems and side effects is endogenous material, therefore one of the patent's semitendinosus tendons is preferred for this procedure.

### Fourth Aspect

According to a forth aspect of the invention, a procedure for the preparation of tendon tissue is provided. The tendon tissue which has been removed from a human or animal body and has a circumference, a longitudinally extending central region, and a first end region having a first free end and a second end region having a second free end. The tendon tissue is prepared with a medical instrument having a needle with a piercing tip and a suture loop at the end of the needle opposite the piercing tip, comprising the steps of:

placing the suture loop around the tendon tissue and pierce the tendon tissue in the medial area with the piercing tip,

after piercing, moving the needle away from the tendon tissue so that when the needle is further moved from the tendon tissue, the loop comes to rest on the circumference of the tendon tissue and the tendon tissue is stabilized by pulling the loop closed,

repeating steps a) and b) at a new puncture site spaced from the previous puncture site by a distance (B) in the direction of the nearby free end of the tendon tissue.

Steps a) to c) are preferably be carried out several times in succession so that the respective end region of the tendon tissue is stabilized up to the respective free end. The whole end region should be stabilized for an optimal handling ability in a later procedure.

To stabilize also the other end region of the tendon tissue, it is advantageous if steps a) to c) are applied several times to both end regions.

It has turned out to be particularly advantageous when the suture loop is knotted after the last piercing in such a way that pulling on the suture does not cause the suture to contract further. The suture loop should not contract any further to avoid damage or separation of the longitudinal fibers of the tendon tissue.

The tendon tissue to be prepared is preferably a tendon tissue part provided by a procedure according to the third aspect of the invention.

Preferably a medical instrument is used for the preparation of the tissue, which comprises a needle having a piercing tip and a thread loop provided at the end of the needle remote from the piercing tip, wherein the thread loop is preferably formed knotless and is attached knotless to the needle. This medical instrument simplifies the preparation procedure.

### DESCRIPTION OF DRAWINGS

The invention will be better understood thanks to the following description and drawings of different embodiments of said invention given as non-limitative examples thereof, wherein:

FIG. 1 shows the incision that is made to access the tendon.

FIG. 2 shows a tape wrapped around the semitendinosus tendon for better handling thereof.

FIG. 3 shows the semitendinosus tendon being divided by a pointed scalpel.

FIG. 4 shows an open tendon stripper being used to mobilize the tendon partly.

FIG. 5 shows a closed tendon stripper being used to mobilize the other part of the tendon.

FIG. 6 shows the residual tendon surrounded by tissue.

FIG. 7 shows the tendon and its different regions characterized for the procedure of preparing the tendon tissue.

FIG. 8 shows an embodiment of a medical device used to prepare the tendon tissue comprising of a thread and a needle where the two ends of the thread are attached to the end of the needle knotless.

FIG. 9 shows the first step of the preparation process when one end of the tendon is pulled through the tread loop.

FIG. 10 shows the tendon tissue in the process of preparation on one end with a loop suture.

FIG. 11 shows the tendon tissue with one end prepared with a loop suture.

FIG. 12 shows the loop suture schematically.

FIG. 13 shows the first perforation of the cervix.

FIG. 14 shows the second perforation of the cervix.

FIG. 15 shows an embodiment of a bend monopolar spatula used to perforate the cervix.

FIG. 16 shows a straight needle puncturing the peritoneum.

FIG. 17 shows the peritoneum punctured with the thread.

FIG. 18 shows the location where the peritoneum is to be opened for display of the ligamentum longitudinale.

FIG. 19 display of the ligamentum longitudinale.

FIG. 20 shows first slitting of the ligamentum longitudinale.

FIG. 21 shows second slitting of the ligamentum longitudinale.

FIG. 22 shows the forming of a tunnel under the ligamentum longitudinale connecting the first slit and the second slit.

FIG. 23 shows loosening the peritoneum from underlying tissue.

FIG. 24 shows inserting the prepared tendon tissue implant into the first perforation of the cervix.

FIG. 25 shows the prepared tendon tissue implant inserted in first and second perforation and attached to the cervix.

FIG. 26 shows the prepared tendon tissue implant pulled through the preformed tunnel under the ligamentum longitudinale

FIG. 27 shows the side-to-side suture connecting both ends and the other section, located between the cervix and the tunnel, of the prepared tendon tissue implant.

FIG. 28 shows the side-to-side suture schematically connecting both ends and the other section, located between the cervix and the tunnel, of the prepared tendon tissue implant, along with the overall positioning of the implant.

FIG. 29 shows the suture for vaginal wall tightening.

FIG. 30 shows the closing of the peritoneum with a suture.

FIG. 31 shows the closed up peritoneum.

FIG. 32 shows a different embodiment of attaching the first tendon tissue end to the backside of the vagina, and FIG. 31 shows the attachment of the other end of the tendon tissue to the front side of the vagina with a suture.

FIG. 33 shows a different embodiment of attaching the tendon material to the uterus with a suture.

FIG. 34 shows a different embodiment of attaching the tendon tissue to the cervix through perforations thereof under the preservation of the uterus.

FIG. 35 shows the ends of the tendon tissue and pulled through perforations so that the tendon tissue wraps around the cervix like a loop.

FIG. 36 shows the process schematically including the different embodiments

Hereinafter, embodiments of the present invention will be described in detail with reference to the accompanying drawings.

First the standard procedure is described, with the alternative variations later described.

### FIRST EMBODIMENT OF THE INVENTION

In a first embodiment (which is described in the third aspect), a procedure for the harvest of semitendinosus tendon tissue in preparation for getting an implant is described.

First, the operation room is set up in a manner as a cruciate ligament replacement would be performed. General anesthesia is required.

The procedure comprises the following steps:

### Step 1.1:

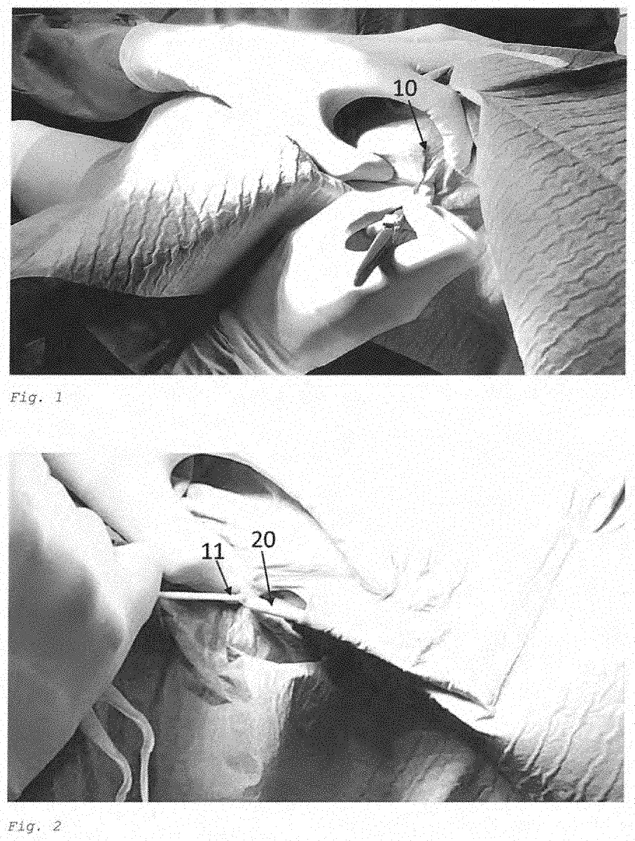

In the medial side of the knee fossa an incision 10 of 2-3 cm is made (FIG. 1) and the semitendinosus tendon 20 is identified. Then a tape 11 is put around the tendon 20 to mobilize it with strong up and down movements (FIG. 2).

### Step 1.2:

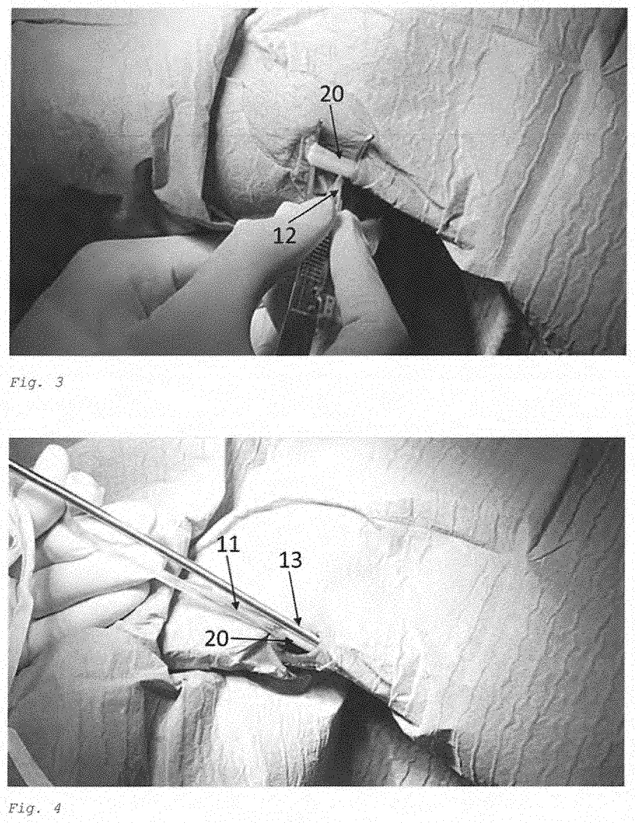

Then a pointed scalpel 12 is used to divide the tendon longitudinal 20 over 5-mm in two parts approximately in a 1/3 to 2/3 ratio, to only harvest the tendon partially (FIG. 3). As the tendon 20 has longitudinal fibers, the division continues until the end of the tendon 20.

### Step 1.3:

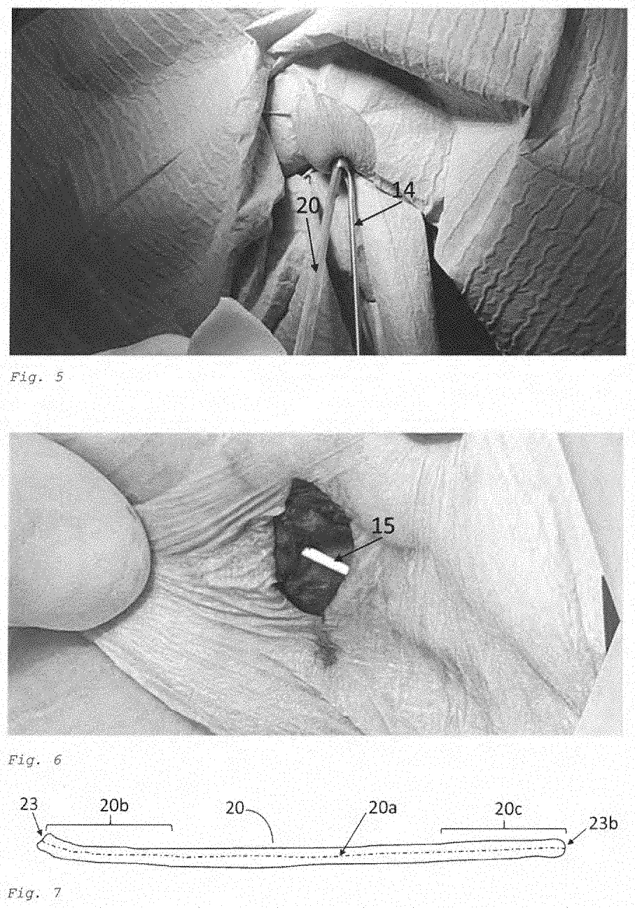

For the division over the whole length, an open tendon stripper 13 is put over the tendon to mobilize the cranial part of the tendon 20 from the muscle and to extend the before made division until the end (FIG. 4). Only one of the parts will be mobilized, this part comprises ideally ⅓ up to ½ of the overall cross-section area of the tendon, but not more than ¾.

### Step 1.4:

The cranial split and mobilized tendon tissue 20 is put through a closed tendon stripper 14 and the caudal part of the tendon tissue is mobilized and finally cut from the pes anserinus (FIG. 5). The tendon tissue can now be removed from the leg.

### Step 1.5:

This is followed by an injection of 20 ml gentamycin (120 mg/200 ml NaCl) along the remaining tendon cranially and caudally. Closure of the skin with two single button sutures and a plaster bandage.

### Step 1.6:

This is followed by partly blunt, partly sharp, removal of muscle tissue remnants on the removed tendon tissue 20 with tweezers and scissors.

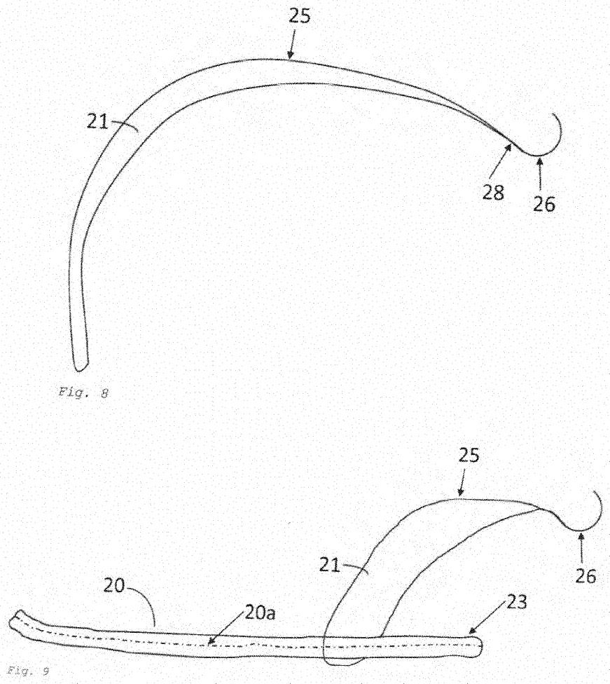

Now the residual part 15 of the tendon is palpable in the knee fossa showing the same tension as before, (FIG. 6) shows the remaining tendon in the incision 10 surrounded by tissue, and thus it continues to provide its function and function of the muscle associated with it. The situation can be compared to a slim and a broad belt under low load. A slim belt can be as effective as a broad belt in holding a trouser.

### SECOND EMBODIMENT OF THE INVENTION

In a second embodiment (which is described in the fourth aspect), a procedure of the preparation of a tendon tissue for implantation into the abdomen is described. The tendon tissue to be prepared is preferably the removed tendon tissue 20, removed according to the first embodiment of the invention.

A workstation with medical preparation equipment, preferably in the same room is set up.

The tendon tissue 20 has a longitudinally extending central region 20a, and a first end region 20b having first free end 23 and a second end region 20c having a second free end 23b (FIG. 7). The tendon tissue ends 23 are prepared with an absorbable loop suture 27 on a separate table. The procedure comprises the following steps:

### Step 2.1:

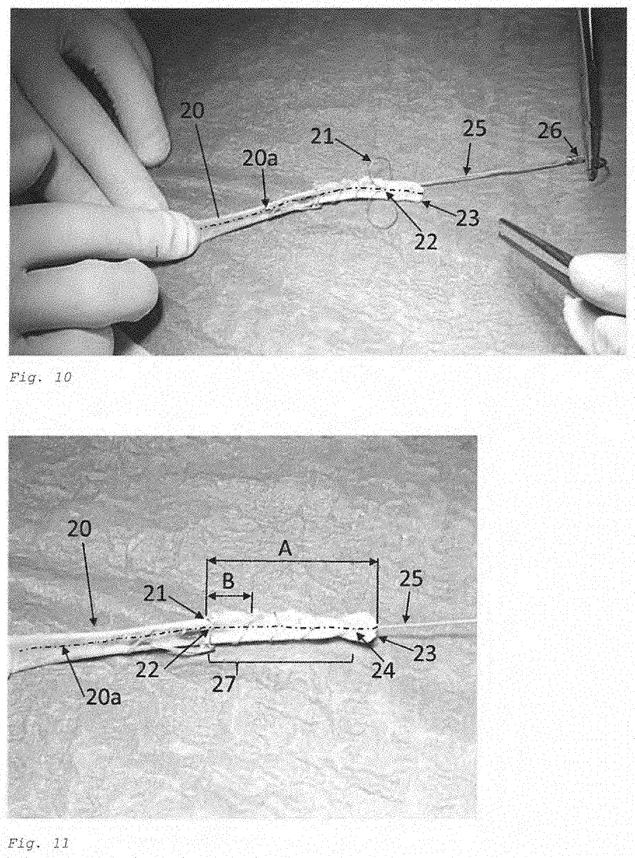

One end 23 of the tendon 20 is pulled through the thread loop 21 of the medical instrument shown in FIG. 8, a double-laid thread 25, (FIG. 9)

### Step 2.2:

and the tendon tissue 20 is pierced with the needle 26 (approx. at the distance of 25% of the tendon length—distance A)

### Step 2.3:

and the thread loop is pulled, by moving the needle away from the tissue, until the thread loop fits tightly to the tendon tissue.

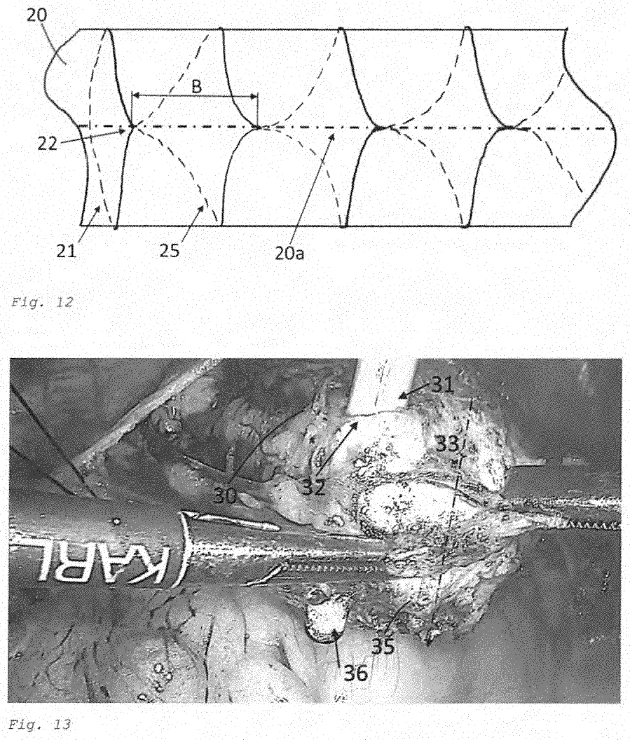

FIG. 10 and FIG. 11 show the preparation during the procedure and one end 23 with the finished loop suture 27.

### Step 2.4:

The end of the tendon 23 is then pulled through the loop 21 again

### Step 2.5:

and the tendon is pierced again ca. 10 mm (distance B) in the direction of the end of the tendon 23.

### Step 2.6:

The thread loop is pulled, by moving the needle away from the tissue, until the thread loop fits tightly to the tendon tissue.

### Step 2.7:

Steps 2.4 to 2.6 are repeated, until the end of the tendon 23 is reached.

### Step 2.8:

At the end of the tendon 23, the loop is knotted 24.

### Step 2.9:

The same procedure is applied to the other end of the tendon 23b.

### Step 2.10:

The prepared tendon tissue 20 is now stored in a moist compress and set aside for later transplantation.

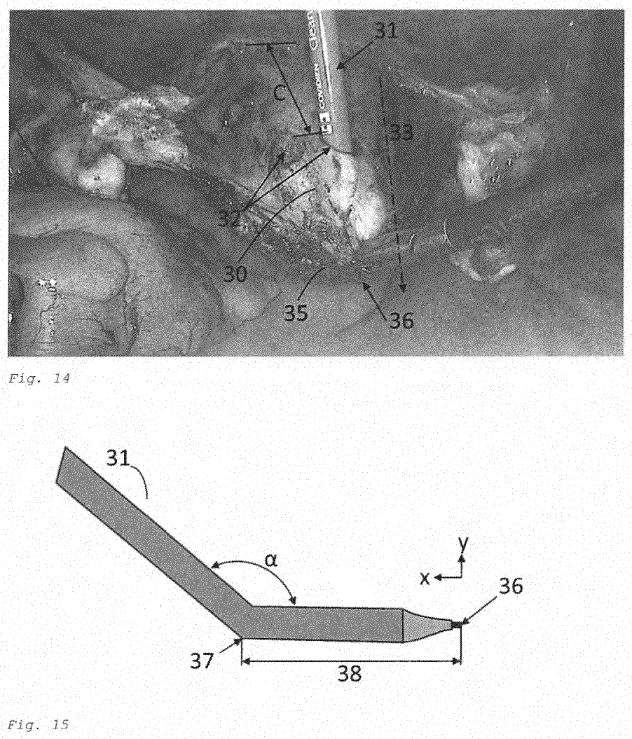

FIG. 12 shows an embodiment of the loop suture 27 in abstracted form. The loop suture prevents the tendon tissue 20 from contracting when the thread 25 is pulled while handling the prepared tissue in further procedures.

A preferred medical instrument for the procedure described in the second embodiment comprises a needle having a piercing tip and a thread loop provided at the end of the needle remote from the piercing tip, as shown in FIG. 8. A thread that has been prepared as a loop 21 is advantageous. Both ends 28 of a thread 25 are attached to the blunt end of a needle 26 and thereby placed in a loop 21. The attachment of the thread 25 to the needle 26 and forming of the thread loop 21 is achieved knotless, that means without knots in the thread 25, so that the thread 25 can be pulled through the tendon 20 without obstruction.

### THIRD EMBODIMENT OF THE INVENTION

In a third an embodiment of the invention (which is described in the first aspect) a procedure for treating pelvic organ prolapse is described, especially for raising the prolapsed organs including vagina, cervix, uterus, bladder, urethra and rectum, with the use of a tendon-type implant made of endogenous or exogenous or bio compatible materials.

The operative room is set up for a standard laparoscopic surgery, preferably, but not exclusively, in the same session as the harvesting of tendon tissue is performed.

The laparoscopy of the patient is now prepared:

The patient is positioned with the upper body down (about 25%—Trendelenburg positioning) so that the bowel follows gravity and allows an optimal view of the internal genitals in the lesser pelvis. The Trendelenburg position is a patient position in the supine position, in which the head assumes the lowest position of the body by inclining the support (surgical table) by 15-30°.

In the navel, the skin is incised longitudinally over 10 mm. There a 10 mm trocar (puncture instrument) is inserted into the navel and the abdomen is filled with CO2 gas (pressure of 15 mmHg).

Now two trocars, each measuring 5 mm, are inserted in the right and left lower abdomen and a trocar measuring 11 mm is inserted centrally directly above the pubic symphysis.

From here on the procedure is performed laparoscopically with instruments preferably, but not exclusively, introduced into the abdomen through the trocars and comprises the following steps:

### Step 3.1:

This is followed by a presentation of the structures in the abdominal cavity. If necessary, adhesions must be detached.

### Step 3.2:

If a supracervical hysterectomy is planned, this is performed now.

Supracervical hysterectomy is the removal of the body of the uterus, leaving the cervix in the body.

### Step 3.3:

After the completion of the supracervical hysterectomy, a large-scale pushing off of the urinary bladder from the cervix and vagina is to be performed.

### Step 3.4:

In order to better define the urinary bladder and not to endanger it, it should be filled with about 50 ml of NaCl.

After pushing off the urinary bladder,

### Step 3.5:

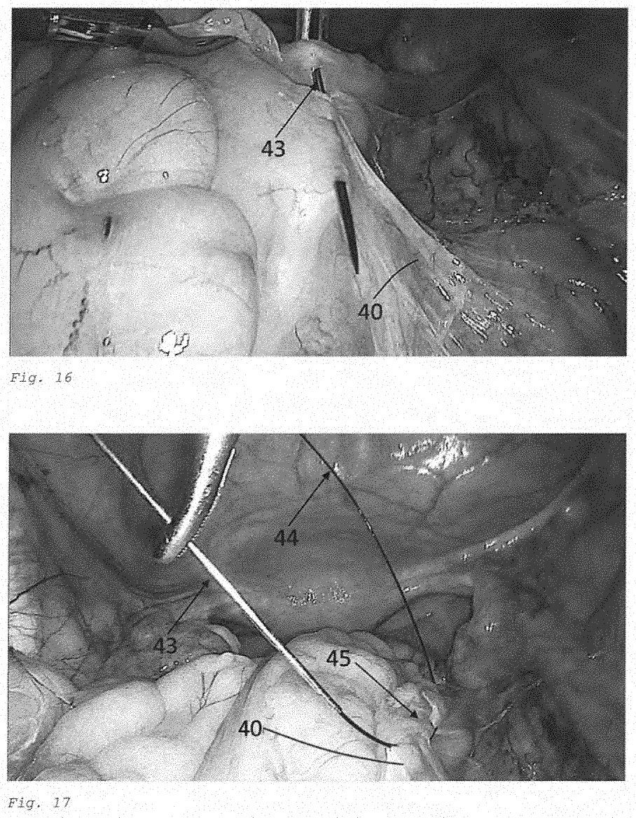

the cervix 30 is perforated twice with a monopolar spatula 31 in the outer half in each case approx. 3 cm (distance C) caudally from ventral to dorsocranial (quasi obliquely upwards towards the back and the head) (direction 33) 32 (FIG. 13 and FIG. 14).

This direction is preferred for safety precautions. The perforations are performed preferably parallel to each other to ensure good tensile force introduction into the cervix later on.

The perforation exit points should be located on the cut face 35 of the cervix 30 to the uterus in order to prevent kinking of the cervix later when pulling on the tendon (FIG. 13 and FIG. 14).

### Step 3.6:

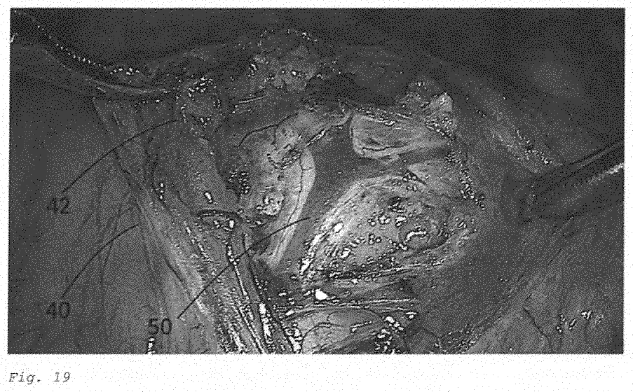

The peritoneum 40, where overlaying the (ossis sacri) Promontorium 41 (FIG. 18) (the anterior upper edge of the 1st sacral vertebra/cruciate bone, protrudes furthest ventrally into the pelvis), is now opened vertically over approx. 5 cm.

### Step 3.7:

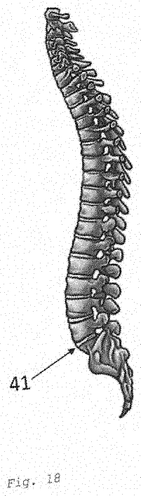

A straight needle 43 (approximately 5 cm long, thread size 0) with monofilament (one fiber and smooth outer) thread 44 is inserted transcutaneously in the left lower abdomen and the opened peritoneum 40 is pierced 3-4 times on the left side at this point 45 (FIG. 16). Then the needle is passed back through the skin to the outside of the abdomen (FIG. 17). By pulling on the thread 44 outside of the abdomen cavity, the peritoneum 40 can be kept open over the promontory 41.

### Step 3.8:

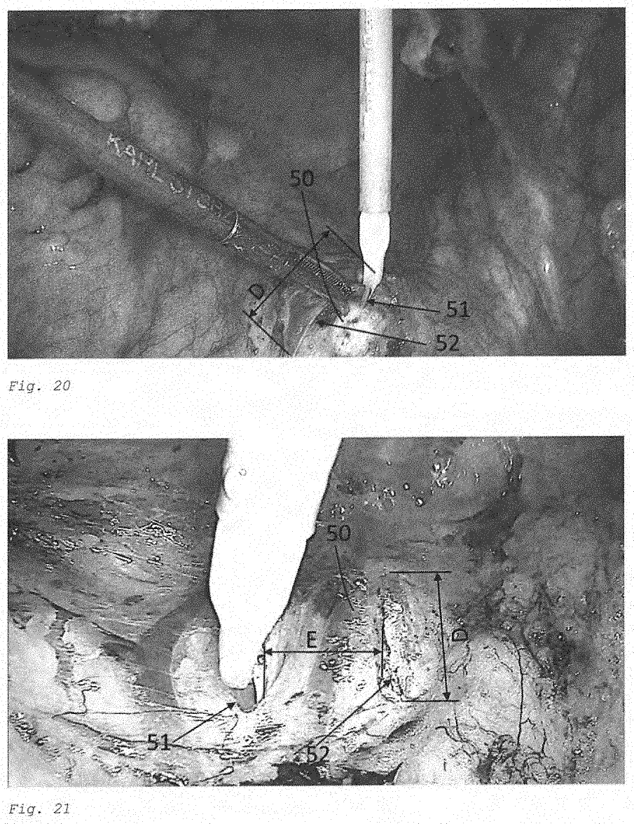

The connective tissue 42 over the longitudinal ligament 50 (ligament that runs along the ventral surface of the entire spine) is pushed aside and the ligament 50 is exposed over a large area. (FIG. 19). Often, blood vessels appear on the longitudinal ligament 50 at the level of the promontory 41. In most cases, they can be left in place and need to be coagulated only in exceptional cases.

### Step 3.9:

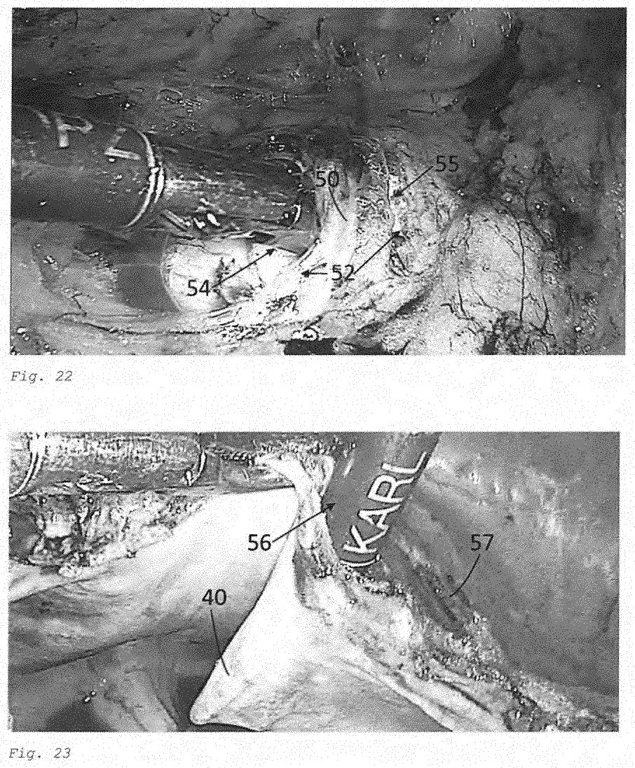

The longitudinal ligament 50, which is longitudinally fibrous, is slit twice in the middle over a length D of about 25 mm, at a distance E of about 10 mm 52 (FIG. 20 and FIG. 21).

A monopolar spatula 51, for example, is suitable for this step.

### Step 3.10:

With the aid of an instrument 54, for example a clamp (Overholt), the ligament 50 is driven under and a kind of tunnel 55 is formed, through which the prepared tendon 20 is later pulled (FIG. 22).

### Step 3.11:

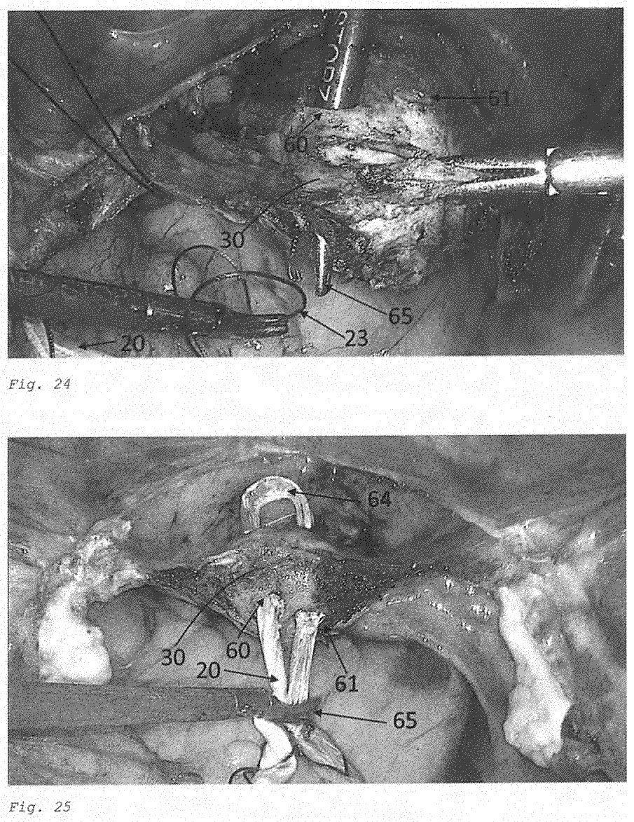

The peritoneum 40 is now undercut on the right side with a blunt instrument 56 and bluntly detached from the underlying tissue 57 in order to feed the tendon 20 through behind it in a following step (FIG. 23). Attention must be paid to the distance to the right ureter and rectum. By bluntly undermining the peritoneum 40 in the area of the cervix posterior wall, this step can be supported from the opposite direction.

### Step 3.12:

Now the implant, preferably the prepared tendon according to the second embodiment, is introduced into the abdominal cavity through the 11 mm trocar.

### Step 3.13:

Using an instrument 65, pass through the left perforation 60 of the cervix and grasp the tendon tissue end 23 and pull through the perforation in caudal direction (FIG. 24). The tendon tissue end is then returned through the right perforation 61 in cranial direction and the cervix is thus stably encircled by the tendon 64 (FIG. 25). As a result, no further fasteners (screws, staples, etc.) are required to attach the tendon tissue to the cervix in this embodiment.

### Step 3.14:

The tendon tissue ends 23 are now passed under the peritoneum 40, where it has been detached from the underlying tissue, to the ligamentum longitudinale 50.

### Step 3.15:

Now a tendon tissue end 23b is pulled through the preformed tunnel 55 under the ligamentum longitudinale (FIG. 26). No further fixation elements are required to connect the tendon tissue to the ligament by using the tunnel 55.

### Step 3.16:

Both ends 23 and 23b of the tendon tissue are pulled taut so that the cervix is lifted from the subsidence position in the cranial direction.

### Step 3.17:

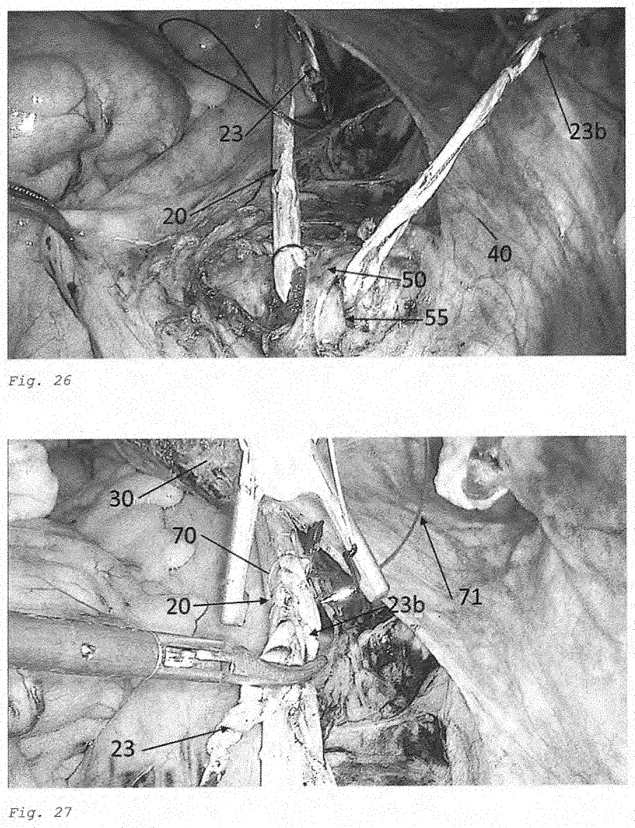

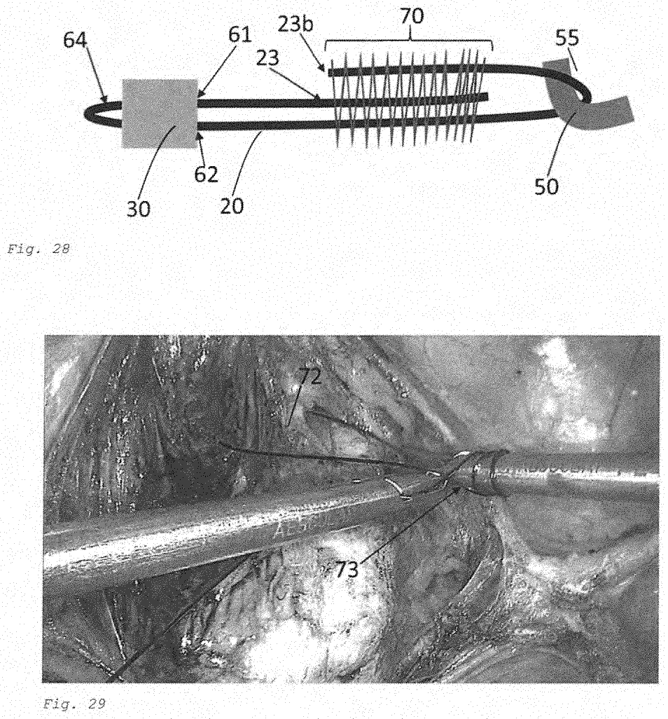

The ends 23 and 23b of the tendon tissue are then sutured side-to-side 70 using non-absorbable suture material 71 (FIG. 27). The side-to-side suture 70 should also comprise the other section of tendon tissue, which runs between the cervix and the ligamentum longitudinale (FIG. 28). As a result, the suture runs through three stacked layers of tendon tissue on top of each other and a stable situation is achieved.

### Step 3.18:

Since there is often also a bulge in the anterior vaginal wall, cystocele, this can also be treated laparoscopically. For this purpose, the bladder is pushed even further caudally from the vagina to the transition from the bladder to the urethra.

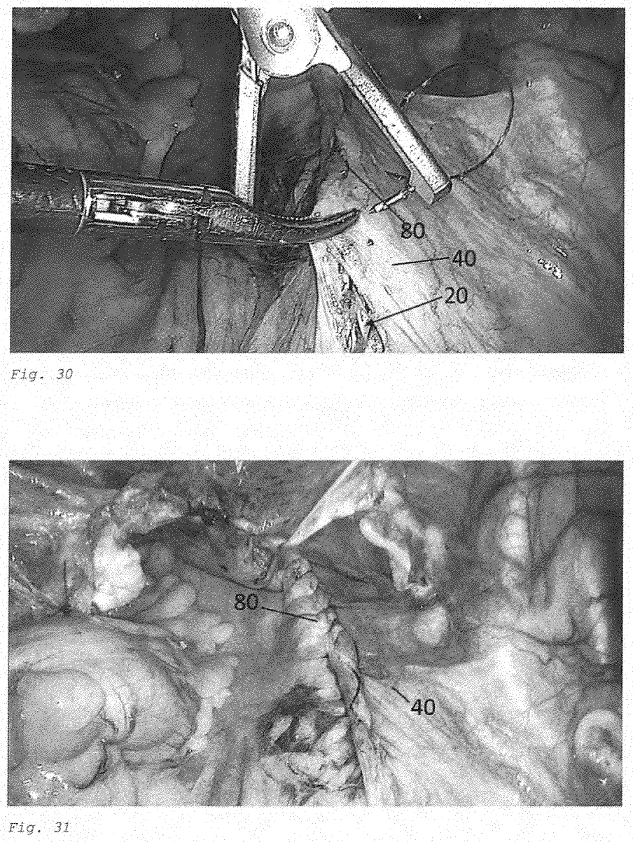

The vaginal wall 72 is then shined from right to left with late absorbable monofilament thread 73 (suitable is for example thread thickness 0) (FIG. 29). This is repeated over approximately four levels cranially to the cervix. Another shining of the vaginal wall follows this from caudal to cranial.

### Step 3.19:

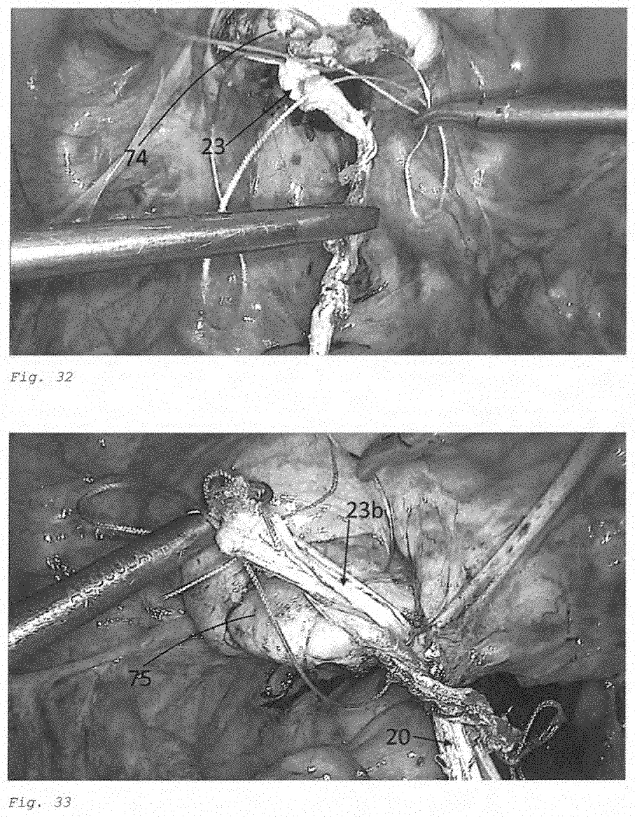

Lastly, there is a continuous closure 80 of the peritoneum 40 (FIG. 30 and FIG. 31). If a supracervical hysterectomy has been performed, the uterus can now be shredded in a bag and extracted.

Alternative 1) for women who have already had hysterectomy with removal of the cervix previous surgery removed, for this procedure, following steps are to be modified:

### Step 2.1.1 to Step 2.10.1:

The tendon tissue is prepared with a non-absorbable suture instead of the absorbable suture. (FIG. 8-FIG. 12).

### Step 3.5.1:

The urinary bladder and rectum are prepared away from the vagina.

### Step 3.13.1:

One end of the tendon tissue 23 is sutured to the back of the vagina 74 (FIG. 32).

### Step 3.15.1:

The other end 23b of the tendon tissue 20 is then pulled through the formed tunnel 55 in the longitudinal ligament 50,

### Step 3.16.1:

then returned to the vagina and pulled taut.

### Step 3.17.1:

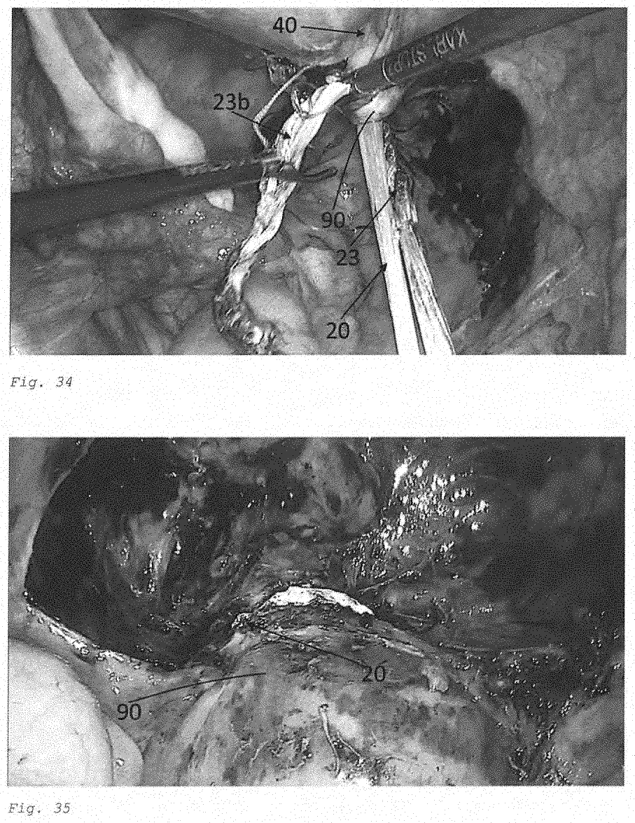

The tendon tissue end 23b is sutured to the front of the vagina 75 (FIG. 33).

Alternative 2) for women who intend to have children in case of uterine prolapse and who wish to preserve their uterus, for this procedure following steps are to be modified:

### Step 3.5.2:

Skipping the perforation of the cervix.

### Step 3.6.2:

For this purpose, the peritoneum 40 is opened at the back of the uterus, at the level of the isthmus.

### Step 3.13.2:

One end of the tendon tissue 23 is sutured, approximately the level of the isthmus, to the back of the uterus 90.

### Step 3.15.2:

The other tendon tissue end 23b is passed through the formed tunnel 55 in the longitudinal ligament 50,

### Step 3.16.2:

and returned to the uterus 90 and pulled taut.

### Step 3.17.2:

After, the other end 23b of the tendon tissue is sutured to the back of the uterus 90. (FIG. 34)

Alternative 3) in older women with a desire to preserve the uterus, in the case of uterine prolapse. This procedure is performed the same as the standard procedure, with not performing step 3.2.

The cervix 30 can also be perforated twice without removing the uterine body 90.

Then the ends of the tendon tissue 23 and 23b are pulled through the perforations 60, 61 so that the tendon tissue 20 wraps around the cervix like a loop (FIG. 35).

Again, a tendon tissue end 23b is pulled through the formed tunnel 55 in the longitudinal ligament 50

and finally the two tendon ends 23, 23b are sutured together “side-to-side” 70.

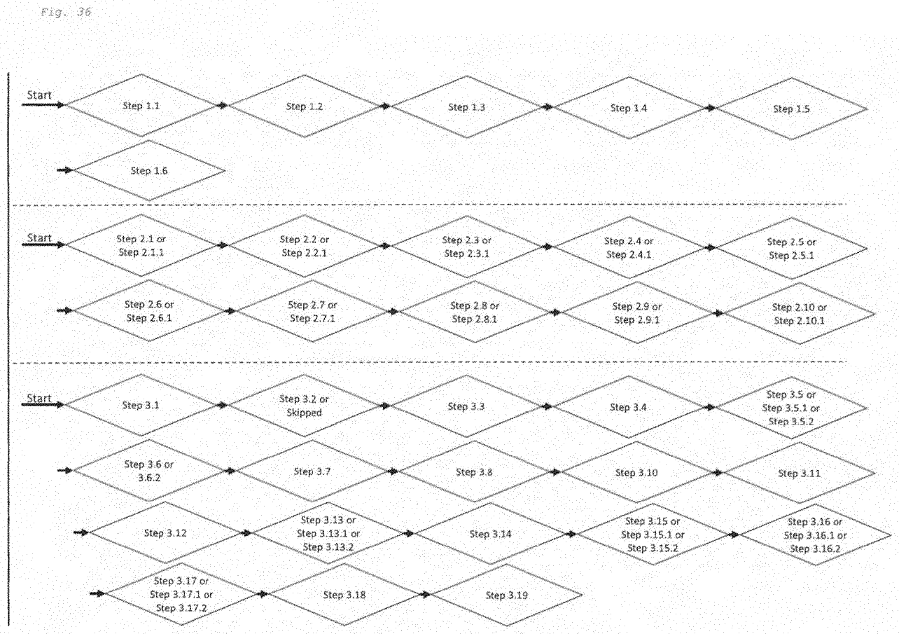

The schematic procedure sequence is illustrated step by step, taking into account the possible alternatives, in FIG. 36.

### FOURTH EMBODIMENT OF THE INVENTION

The fourth embodiment of the invention (which is described in the second aspect) relates to a medical instrument comprising a monopolar spatula 31 with a kink 37. The spatula 31 used for perforating the cervix in the fourth embodiment should comprise a kink 37 with an angle α, of about 135°, at a distance 38 in the x-direction, here about 3 to 4 cm, in front of the stripped cutting end 36 (FIG. 15). The width of the cutting end 36 in z-direction (z-direction is the direction that is perpendicular to the x-direction and the y-direction) is preferably 4 mm. The thickness of the cutting end 36 in y-direction is preferably 2 mm. The instrument is preferably configured such that when inserted into the patient, the instrument is aligned along a straight axis and does not comprise the kink 37. Further, the instrument is configured such that when the spatula with the cutting end 36 is inserted into the body, the kink 37 is provided. The cutting end 36 of the instrument is preferably of a round shape, which preferably runs along a circular line, when viewed from above (y-direction) (FIG. 13). This instrument enables the perforations in described shape and routing, as the entry point of the perforation in the cervix is not reachable with a straight spatula.

1. A procedure for repairing and treating pelvic prolapses in a female patient, comprising the following steps:
a) providing an elongated tendon-type implant having a first end and a second end,
b) attaching the first end of the implant to the longitudinal ligament of the patient,
c) attaching the second end of the implant to the pelvic organ of the patient,
d) wherein the attachment of the implant is to be made under tension so that the pelvic organ is raised from the prolapsed position to a normal position.

2. The procedure according to claim 1,
wherein the attaching of the first end and/or the second end of the implant according to step b) and c) is performed by fastening elements.

3. The procedure according to claim 1,
wherein a tunnel with a tunnel entrance and a tunnel exit is formed in or around the longitudinal ligament in the region of the promontory ossis sacri, and
wherein the attachment according to step b) is performed by passing one end of the implant through the tunnel.

4. The procedure according to claim 1,
wherein the tunnel is inserted so that the tunnel entrance and the tunnel exit are provided on the side of the longitudinal ligament facing away from the promontory ossis sacri.

5. The procedure according to claim 1,
wherein at least one perforation is made in the cervix, and
wherein the attaching to the cervix according to step c) is performed by passing one end of the implant through the perforation.

6. The procedure according to claim 5,
wherein the perforation is performed in direction caudal from ventral to dorsocranial.

7. The procedure according to claim 6, wherein two parallel or almost parallel perforations are performed, wherein each perforation have a cranial and a caudal opening, and
wherein the attaching to the cervix according to step c) is performed as follows:
inserting one end of the implant through the cranial opening of the first perforation in direction to the caudal opening and passing in caudal direction through the caudal opening, after exiting the caudal opening of the first perforation inserting the end of the implant into the caudal opening of the second perforation and passing in cranial direction through the cranial opening.

8. The procedure according to claim 1, wherein the ends of the implant are pulled together to provide the tension according to step d).

9. The procedure according to claim 8, wherein the ends of the implant are sutured together under tension.

10. The procedure according to claim 1, wherein the implant is made of biocompatible material and/or of biological material.

11. The procedure according to claim 1, wherein the implant is composed from prepared tendon tissue from the semitendinosus tendon.

12. The procedure according to claim 1, wherein the procedure is performed laparoscopically.

13. Medical instrument for perforation of the cervix, said instrument being formed as a monopolar spatula and having a kink at a distance X from a cutting end.

14. The instrument according to claim 13, wherein the kink encloses an angle α in the range of 90° to 175°.

15. Procedure for removing tendon tissue of a tendon from a human or animal body, comprising the following steps:
a) exposing a tendon present in the human or animal body,
b) dividing the tendon after exposing it in the direction of its longitudinal fibers into two elongated portion of tendon tissue,
c) separating and removing one of the two portions of tendon tissue; and
d) remaining the other portion of the tendon tissue in the body.

16. The procedure according to claim 15, wherein the removed tendon tissue portion comprises ¼ to ¾ of the cross-sectional area of the exposed tendon.

17. The procedure according to claim 15, wherein dividing the exposed tendon according to steps b) is first performed by slitting the tendon and then dividing the slitted tendon with a tendon stripper.

18. The procedure according to claim 15, wherein the exposed tendon is the semitendinosus tendon and the removed tendon tissue portion is tissue of the semitendinosus tendon.

19. A procedure for the preparation of tendon tissue,
wherein the tendon tissue has been removed from a human or animal body and has a circumference, a longitudinally extending central region, and a first end region having a first free end and a second end region having a second free end,
wherein the tendon tissue is prepared with a medical instrument having a needle with a piercing tip and a suture loop at the end of the needle opposite the piercing tip, comprising the steps of:
a) placing the suture loop around the tendon tissue and piercing the tendon tissue in the medial area with the piercing tip,
b) after piercing, moving the needle away from the tendon tissue so that when the needle is further moved from the tendon tissue, the loop comes to rest on the circumference of the tendon tissue and the tendon tissue is stabilized by pulling the loop closed, and
c) repeating steps a) and b) at a new puncture site spaced from the previous puncture site by a distance (B) in the direction of the nearby free end of the tendon tissue.

20. The procedure according to claim 19, wherein steps a) to c) are carried out several times in succession so that the respective end region of the tendon tissue is stabilized up to the respective free end.

21. The procedure according to claim 19, wherein steps a) to c) are applied several times to both end regions.

22. The procedure according to claim 19, wherein the suture loop is knotted after the last piercing in such a way that pulling on the suture does not cause the suture to contract further.

23. The procedure according to claim 19, wherein the tendon tissue to be prepared is a tendon tissue part provided by a procedure comprising the steps of:
i. exposing a tendon present in the human or animal body,
ii. dividing the tendon after exposing it in the direction of its longitudinal fibers into two elongated portion of tendon tissue,
iii. separating and removing one of the two portions of tendon tissue; and
iv. remaining the other portion of the tendon tissue in the body.

24. A medical instrument comprising a needle having a piercing tip and a thread loop provided at the end of the needle remote from the piercing tip.

25. The medical instrument according to claim 24, wherein the thread loop is formed knotless and is attached knotless to the needle.
